(19) United States
(12) Patent Application Publication
(10) Pub. No.: US 2024/0164996 A1
(43) Pub. Date: May 23, 2024

(54) DENTAL MATERIAL FOR THE PREPARATION OF FULL OR PARTIAL DENTAL PROSTHESES

(71) Applicant: Ivoclar Vivadent AG, Schaan (LI)

(72) Inventors: Christoph Haydl, Rheineck (CH); Francois Loiseau, Messen (CH); Clemens Andreas Häfele, Feldkirch (AT); Petra Dellafior, Feldkirch (AT)

(21) Appl. No.: 18/503,482

(22) Filed: Nov. 7, 2023

(30) Foreign Application Priority Data
Nov. 8, 2022 (EP) 22206261.4

(51) Int. Cl. A61K 6/893; A61C 13/00; A61C 13/01; A61C 13/087; A61C 13/10; A61K 6/76; A61K 6/79

(52) U.S. Cl. CPC A61K 6/893; A61C 13/0013; A61C 13/0019; A61C 13/01; A61C 13/087; A61C 13/1003; A61K 6/76; A61K 6/79

(57) ABSTRACT

A radically polymerizable material for the preparation of dental prostheses, containing (a) at least one monofunctional radically polymerizable (meth)acrylate, (b) at least one polyfunctional radically polymerizable (meth)acrylate, (c) at least one ABA triblock copolymer, (d) at least one nanoparticulate or microfine filler having an average primary particle size of from 10 nm to 350 nm, and (e) at least one photoinitiator. The A block of the ABA triblock copolymer is an oligomeric polyester and the B block is an oligomeric polysiloxane.

### CROSS-REFERENCE TO RELATED APPLICATIONS

This application claims priority to European patent application No. EP22206261.4 filed on Nov. 8, 2022, the disclosure of which is incorporated herein by reference in its entirety.

### TECHNICAL FIELD

The present invention relates to radically polymerizable dental materials which are suitable for the preparation of the denture base and the replacement teeth of full or partial dental prostheses. The materials are particularly suitable for processing with 3D printing methods.

### BACKGROUND

Full and partial dental prostheses serve as a replacement for one or more missing teeth and consist of the denture base and the replacement teeth. The denture base rests at least partially on or against the oral mucosa and holds the replacement teeth. Full and partial dental prostheses are usually removable. Full dental prostheses are also referred to as full dentures and partial dental prostheses as partial dentures.

The mechanical requirements for the denture base and the replacement teeth differ considerably, and consequently different materials are used to prepare the denture base and the replacement teeth. This is associated with a number of disadvantages. One problem is creating a strong bond between the materials, which is more challenging with different materials than with uniform materials. Another problem is that different materials usually require different processing methods, making it more difficult to prepare dentures. Additive manufacturing processes can be used to prepare dentures, such as stereolithography and inkjet printing. In additive manufacturing processes, which are also known as generative manufacturing processes, 3D shaped bodies are created layer by layer from polymerizable materials based on a CAD data set, with the layers being cured by controlled exposure to light. Inkjet printing processes offer the advantage over stereolithography that several materials can be processed in the same additive process. However, this requires a certain similarity and compatibility of the materials so that they can be processed together with one apparatus.

A further problem lies in the worldwide trend of steadily tightening regulatory requirements for the approval of new materials for medical applications. From this point of view, uniform materials containing a small number of chemically different components are advantageous because they facilitate approval.

EP 2 492 289 A1 discloses dental materials containing block copolymers, radically polymerizable monomers and a polymerization initiator. The block copolymers comprise hard segments of methacrylic acid esters and soft blocks of acrylic acid esters. The hard blocks are characterized by a high glass transition temperature and the soft blocks by a low glass transition temperature. The materials are relatively flexible so that they do not break under stress, but they have a low flexural modulus. They are said to be particularly suitable as dental cements.

WO 2014/078537 A1, which is hereby incorporated by reference, discloses resin mixtures for the preparation of dental shaped bodies by 3D printing processes based on mono- and multifunctional methacrylates, which comprise silicone-acrylate based impact modifiers with core-shell structure for improving impact strength and fracture toughness. These silicone acrylate-based modifiers tend to discolor and to float or sediment on the monomer mixture, which is disadvantageous.

US 2018/0000570 A1, which is hereby incorporated by reference, relates to fabricating materials based on mono- and multifunctional (meth)acrylates for the additive manufacturing of dental components. The fabricating materials contain silicone-acrylate based rubber particles with a core-shell structure (product S2006, Mitsubishi Rayon Co.) as impact modifiers and oligomers prepared by reacting trimethyl 1,6-diisocyanate, bisphenol A propoxylate and 2-hydroxyethyl methacrylate (HEMA). The cured components are said to have good mechanical and physical properties as well as a good biocompatibility.

U.S. Pat. No. 10,299,896 B2 and US 2019/0053883 A1, which are hereby incorporated by reference, disclose dental components produced by additive processes comprising at least two layers of building materials with different compositions. One layer is formed by a material comprising oligomers which are obtained by reacting intermediate products having terminal isocyanate groups with hydroxyl-based methacrylates, a polymerizable acrylic compound, and an impact modifier. At least one further layer is formed by a material which contains a urethane monomer, a glycol dimethacrylate and filler. The combination of materials with different mechanical and physical properties is said to be advantageous for adapting the components to different requirements. Commercially available polymers with a core-shell structure, such as the product M570 from Kaneka, are used as impact modifiers.

EP 3 564 282 A1 discloses curable compositions for high temperature photopolymerization processes which contain an oligomeric urethane dimethacrylate as a glass transition temperature modifier, a (poly)carbonate (poly)urethane dimethacrylate as a toughness modifier and optionally core-shell particles. They are said to have good thermomechanical properties and biocompatibility and to be suitable for the production of orthodontic appliances.

US 2020/0231803 A1, which is hereby incorporated by reference, discloses curable compositions comprising at least one monofunctional acrylate, a multifunctional acrylate and PPG-PEG-PPG or PPG-PEG-PPG block copolymer acrylate, a multifunctional acrylate and PPG-PEG-PPG or PPG-PEG-PPG block copolymer.

U.S. Pat. No. 11,028,204 B2, which is hereby incorporated by reference, discloses radically polymerizable compositions which comprise at least one (meth)acrylate monomer or oligomer and at least one monofunctional (meth)acrylate monomer having a polycyclic group that contains at least three rings. The compositions are said to be characterized by high toughness, tensile strength and tensile elongation and to be suitable, inter alia, to be used as inks for three-dimensional inkjet printing for stereolithography.

The materials known thus far are only suitable to a limited extent for the preparation of dental prostheses, i.e., of the denture base and replacement teeth. They require concessions with regard to the properties of the denture base or the replacement teeth or represent a compromise which is not optimal for any of the denture components.

### SUMMARY

The object of the invention is to provide radically polymerizable materials which are suitable for preparing both the denture base and the replacement teeth. The materials should be processable by additive processes, in particular by stereolithography and quite particularly by inkjet printing processes.

### DETAILED DESCRIPTION

According to the invention, this object is solved by radically polymerizable materials which contain
(a) at least one monofunctional radically polymerizable (meth)acrylate, preferably acrylate,
(b) at least one polyfunctional radically polymerizable (meth)acrylate, preferably methacrylate,
(c) at least one block copolymer,
(d) at least one nanoparticulate or microfine filler having an average primary particle size from 10 nm to 350 nm, and
(e) at least one photoinitiator.

The dental materials according to the invention are characterized in that they contain at least one ABA triblock copolymer as block copolymer (c).

Block copolymers are macromolecules consisting of two or more homopolymer blocks which are covalently bound with each other. The materials are preferably characterized in that the A block of the ABA triblock copolymer is an oligomeric polyester, in particular an oligomeric polycaprolactone (PCL), and the B block is an oligomeric polysiloxane, in particular a PDMS.

Preferred block copolymers according to the invention can be prepared using the known methods of living or controlled polymerization, for example by radical or ionic (anionic and cationic) polymerization, with controlled radical polymerization and living anionic polymerization being preferred. However, block copolymers can also be obtained by coupling of suitable end groups of homopolymers.

ABA block copolymers can be prepared, for example, by preparing a B block through anionic polymerization of monomer B via a dianion mechanism. The B mid-block formed carries an anion end group on each side, which initiates the anionic polymerization of monomer A, forming the two A blocks (method 1). Alternatively, ABA triblock copolymers can be obtained by esterification of a telechelic B block, which carries at both ends a suitable functional group, e.g., an OH group, with two A blocks which are functionalized only on one side, e.g., with a COOH group (Method 2). Finally, OH-telechelic homopolymers of monomer B can be esterified with α-bromoisobutyric acid. The two thus-formed bromine end groups in homopolymer block B can then be utilized as starting center for the formation of the two A blocks by ATRP (method 3).

The monomers used to prepare the block copolymers are preferably selected such that the A blocks are miscible with the resin matrix, i.e. the mixture of components (a) and (b), and the B block is not homogeneously miscible with the resin matrix.

Here, the miscibility is understood in terms of thermodynamics with respect to the single phase state. Accordingly, a miscible polymer block is understood to be a polymer block consisting of a monomer, the homopolymer of which is soluble in the resin matrix with the result that the mixture has a transparency of at least 95%. In contrast, if the mixture is cloudy or opaque, i.e. the transparency is lower than 95%, then the homopolymer and thus the corresponding polymer block is not miscible with the resin matrix. The transparency is measured in accordance with the ISO 10526:1999 standard with a spectrophotometer on 1 mm thick test specimens polished to a high gloss in transmission (D65), e.g. with a CM-5-type Konika-Minolta spectrophotometer.

The block copolymers cause a significant improvement in the fracture toughness of the materials according to the invention after curing. It is assumed that the immiscibility of the B blocks of the block copolymers with the other constituents of the compositions according to the invention brings about a microphase separation and thus the formation of morphologies at the nanoscale level. Here, the macromolecules of the ABA block copolymers form spherical or worm-like phases in the monomer resin or, during curing, by self-assembly, which phases can interact with crack tips, that is to say crack tips come into contact with the phases and the fracture energy is distributed to the phases such that the cracks do not migrate further through the material and do not increase in size. The propagation of a crack can be observed in transparent materials under an electron microscope. In fracture mechanics, the frontmost part of the crack is called the crack tip.

In a preferred embodiment, the dental material according to the invention contains a silicon-containing ABA triblock copolymer as component (c).

ABA triblock copolymers are “silicon-containing” for the purposes of the present invention if they have at least one Si atom in the backbone or side chain, preferably in the backbone. It is preferred that at least one block contains a polysiloxane oligomer. Particularly preferably, the B block is silicon-containing and, more preferably, a polysiloxane oligomer.

The A block is preferably a polyester oligomer or a poly(meth)acrylate. A particularly preferred poly(meth)acrylate is polymethyl methacrylate (PMMA). A particularly preferred example of a polyester oligomer is a polylactone oligomer and even further preferred a polymer of caprolactone. The A block is thus preferably a polycaprolactone (PCL) oligomer.

In the case that the A block is a poly(meth)acrylate, the A block has a molar mass of 0.7 to 7 kDa, preferably 1 to 5.5 kDa, and particularly preferably 1.5 to 4 kDa. In another preferred embodiment, the A block is a poly(meth)acrylate and the molar mass of the total ABA block copolymer is less than 10 kDa, in particular less than 7 kDa. The B block is preferably a polyolefin, in particular a polyethylene, or a polysiloxane oligomer, particularly preferably a polysiloxane oligomer in accordance with the formula —O—(SiR₂—O)ₚ, in which
R is a linear C₁-C₂₀ alkyl, branched C₃-C₁₂ alkyl or C₆-C₂₀ aryl group, wherein the individual R radicals can be identical or different, and
P is a number from 3 to 100, preferably a number from 10 to 50.

Quite preferably, the B block is a polymer of dimethylchlorosilane, cyclotri- or cyclotetradimethoxysilane. The B block is particularly preferably a poly(dimethylsiloxane) (PDMS) oligomer.

The B blocks are characterized by a relatively high flexibility. By flexible blocks is meant blocks which are formed from monomers, the homopolymers of which have a glass transition temperature TG below 50° C., preferably below 0° C., and particularly preferably in the range from −30 to −110° C. Block copolymers with flexible blocks improve the fracture toughness without significantly affecting the flexural strength and the modulus of elasticity of the polymers.

Polyester-polysiloxane block copolymers according to the following general formula I are preferred according to the invention:

(PCL)_{q}-b-(PDMS)ᵣ-b-(PCL)_{q} Formula I

in which
q in each case is a number from 5 to 40, preferably from 10 to 20, and
r is a number from 10 to 100, preferably from 30 to 60.

(PCL)_{q} represents polycaprolactone, which is composed of q caprolactone monomers, and (PDMS)ᵣ represents poly(dimethylsiloxane), which is composed of r dimethylsiloxane monomers. The letter b represents block.

Further preferred are poly(meth)acrylate-polysiloxane block copolymers which contain a polymethyl methacrylate radical as A block and a polysiloxane radical as B block, wherein the polysiloxane radical is preferably as defined above and is particularly preferably a poly(dimethylsiloxane) radical.

Particularly preferably, the ABA triblock copolymers PCL-b-PDMS-b-PCL and PMMA-b-PDMS-b-PMMA have a molar ratio A:B of 0.1 to 5 and a molar mass preferably of 3 to 25 kDa, particularly preferably 4 to 20 kDa and quite particularly preferably 5 to 10 kDa. A preferred block copolymer is PCL-b-PDMS-b-PCL, wherein the PDMS blocks have a molar mass of about 3200 g/mol and the PCL blocks each have a molar mass of about 1600 g/mol. In a preferred embodiment, the molar mass of each of the A blocks is 40-60% and preferably 50% of the molar mass of the B block. In another preferred embodiment, the chain length of the A blocks is in each case 40-65% and preferably 45-60% of the chain length of the B block.

Preferably, the composition of the block copolymers is selected to contain 60:40 to 40:60 wt.-% A blocks and 40:60 to 60:40 wt.-% of the B block, based on the total weight of the block copolymer.

The block copolymer or the block copolymers are preferably used in an amount of 0.5 to 12% wt.-%, more preferably in an amount of 1 to 10 wt.-% and most preferably 2 to 8 wt.-%, based on the total weight of the dental material.

It was found that the block copolymers used according to the invention significantly improve the fracture toughness of the polymer networks and also opacify the materials. As a result, the amount of pigments used for opacification can be reduced. In addition, they bring about only a relatively small increase in viscosity. A further advantage of the block copolymers used according to the invention is that they can easily be homogeneously mixed with the remaining components of the materials, whereas the homogeneous dispersion of core-shell polymer particles is much more complex. In addition, particles tend to sediment, with the result that compositions based on core-shell particles are less stable. In contrast, the block copolymers used according to the invention can be readily incorporated into resin mixtures, with the result that it is possible to match the materials to the intended application and to set the desired fracture toughness and fracture work without problems.

The dental materials according to the invention preferably contain a monofunctional, non-aromatic alicyclic, bicyclic or tricyclic (meth)acrylate as component (a), wherein acrylates are particularly preferred.

Preferred alicyclic meth(acrylates) are compounds of the following formula,

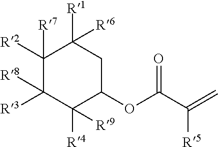

in which
R′¹ to R′⁴ are independently selected from the group consisting of H and C₁-C₆ alkyl, preferably H and C₁-C₄ alkyl, particularly preferably H, methyl, ethyl and isopropyl,
R′⁵ is H or methyl,
R′⁶ to R′⁹ are independently selected from H and C₁-C₃-alkyl, with H being particularly preferred.

Particularly preferred examples of an alicyclic component (a) are 4-tert-butylcyclohexyl (meth)acrylate, 2-isopropyl-5-methylcyclohexyl (meth)acrylate, 3,3,5-trimethylcyclohexyl (meth)acrylate and cyclohexyl methacrylate.

Non-aromatic bi- and tricyclic (meth)acrylates and in particular acrylates are particularly preferred. Preferred examples are compounds of formula II:

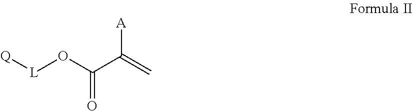

in which
A is H or methyl,
L is a chemical bond, C₁-C₃-alkylene or —(CR¹R²—CR³R⁴—O)ₘ—, preferably a chemical bond or —CH₂—, wherein R¹ to R⁴ are each independently H or a C₁-C₃ alkyl group, preferably H, and m is an integer from 2 to 10, preferably 2, and
Q is an isobornyl derivative or a norbornyl derivative, preferably an unsubstituted isobornyl or an isobornyl substituted with one or more C₁-C₃ alkyl groups or a unsubstituted norbornyl or a norbornyl substituted with one or more C₁-C₃ alkyl groups, in Q particular is one of the following groups in which • represents the point of linkage to L:

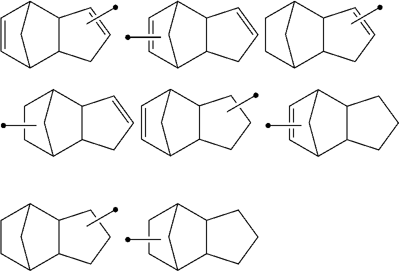

Particularly preferably, the compound of formula II is a mono(meth)acrylate of tricyclodecane methanol, tricyclodecane dimethanol or dicyclopentadienyl.

Particularly preferred are the mono(meth)acrylates of tricyclodecane methanol having the following formulas in which A is an H atom or methyl:

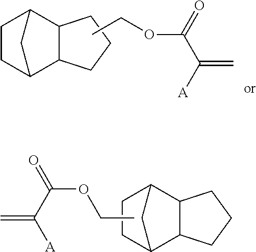

Most preferred is the tricyclopentanyl acrylate having the following formula III:

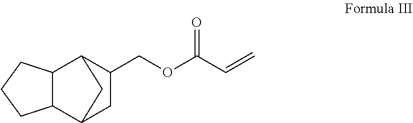 [SMILES: C=CC(=O)OCC1CC2CC1C1CCCC21]

This is also referred to as tricylodecane methanol monoacrylate (TCDMA).

Mixtures of isomers of compounds of formulas II/III can also be used, i.e. the isomers can differ in that the —CH₂—O-acrylate group is linked to the tricyclic group at different positions.

The dental materials according to the invention preferably contain as component (b) at least one polyfunctional urethane acrylate or preferably urethane methacrylate, particularly preferably a urethane dimethacrylate.

Polyfunctional radically polymerizable compounds are compounds with two or more, preferably 2 to 4 and in particular 2 radically polymerizable groups. Accordingly, monofunctional monomers have only one radically polymerizable group. Polyfunctional monomers have crosslinking properties and are therefore also referred to as crosslinking monomers.

Preferred difunctional urethane dimethacrylates (b) are urethanes of 2-(hydroxymethyl) acrylic acid methyl ester and diisocyanates, such as 2,2,4-trimethyl hexamethylene diisocyanate or isophorone diisocyanate, tetramethylxylylene diurethane ethylene glycol di(meth)acrylate or tetramethylxylylene diurethane-2-methylethylene glycol di(meth)acrylate (V380) and particularly preferably UDMA (an addition product of 2-hydroxyethyl methacrylate and 2,2,4-trimethylhexamethylene-1,6-diisocyanate). Component (b) is, just like component (a), a monomer. The dental materials according to the invention preferably do not contain any radically polymerizable oligomers, in particular no oligomeric urethane dimethacrylates.

The monomer tetramethylxylylene diurethane ethylene glycol di(meth)acrylate or tetramethylxylylene diurethane 2-methylethylene glycol diurethane di(meth)acrylate (V380) has the following formula:

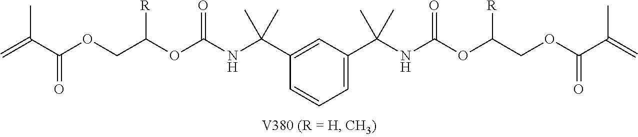

In the formula shown, the R groups are each independently H or CH₃, and the groups can have the same meaning or different meanings. Preferably, a mixture is used containing molecules in which both groups are H, molecules in which both groups are CH₃, and molecules in which one group is H and the other group is CH₃, wherein the ratio of H to CH₃ is preferably 7:3. Such a mixture can be obtained, for example, by reacting 1,3-bis(1-isocyanato-1-methylethyl)benzene with 2-hydroxypropyl methacrylate and 2-hydroxyethyl methacrylate.

The materials according to the invention can contain two or more different polyfunctional radically polymerizable (meth)acrylates (b), particularly preferably at least one polyfunctional urethane (meth)acrylate and optionally a further polyfunctional (meth)acrylate.

Preferred further di- and polyfunctional (meth)acrylates are bisphenol A dimethacrylate, Bis-GMA (an addition product of methacrylic acid and bisphenol A diglycidyl ether), ethoxy- or propoxylated bisphenol A dimethacrylates, such as the bisphenol A dimethacrylate SR-348c (Sartomer) having 3 ethoxy groups or 2,2-bis[4-(2-methacryloyloxypropoxy)phenyl]propane, di-, tri- or tetraethylene glycol dimethacrylate, trimethylolpropane trimethacrylate, pentaerythritol tetramethacrylate and glycerol di- and trimethacrylate, 1,4-butanediol dimethacrylate, diol(meth)acrylate compounds, in particular 1,10-decanediol dimethacrylate (D₃MA), bis(methacryloyloxymethyl)tricyclo-[5.2.1.0^{2,6}]decane (DCP), polyethylene glycol dimethacrylates or polypropylene glycol dimethacrylates, such as polyethylene glycol 200-dimethacrylate or polyethylene glycol 400-dimethacrylate (PEG-200- or PEG-400-DMA) or 1,12-dodecanediol dimethacrylate. Preferred further polyfunctional (meth)acrylates include decanediol dimethacrylate, ethylene glycol dimethacrylate (EGDMA) and butane-1,4-diol dimethacrylate (BDDMA).

The materials according to the invention can contain up to 10 wt.-%, preferably up to 5 wt.-%, of further polyfunctional (meth)acrylates. Particularly preferred are materials which contain exclusively one or more urethane di(meth)acrylates as component (b) and no further di(meth)acrylates.

Preferably, the glass transition temperature TG of homopolymers of component (a) is 25° C. or more and that of homopolymers of component (b) is 110° C. or more. It is further preferred that the difference in glass transition temperatures of homopolymers of the components (b) and (a) is at least 40° C. Glass transition temperatures can be determined in accordance with ISO 11357-1:2016.

In a particularly preferred embodiment, component (a) is an acrylate and component (b) is a methacrylate. It is further preferred that the material according to the invention contains exclusively acrylates as component (a) and exclusively methacrylates as component (b).

The dental materials according to the invention preferably contain an inorganic particulate filler as nanoparticulate or microfine filler (d). Preferred inorganic fillers are oxides, such as SiO₂, ZrO₂ and TiO₂ or mixed oxides of SiO₂, ZrO₂, ZnO and/or TiO₂, particularly preferably fumed silica or precipitated silica. The fillers have a primary particle size of 10 to 350 nm, preferably 10 to 200 nm and particularly preferably 20 to 100 nm. Particularly preferred fillers are mixed oxides of SiO₂ and ZrO₂, precipitated silica or most preferably fumed silica.

In a preferred embodiment, the fillers have an average primary particle size (d₅₀) of 50 to 350 nm, in particular 100 to 300 nm and particularly preferably 150 to 250 nm, measured by means of dynamic light scattering (DLS).

The filler or the fillers (d) function as rheology modifiers, serve to increase hardness and modulus of elasticity and to improve abrasion resistance. Fillers with small particle size have a greater thickening effect than fillers with larger particles.

According to the invention, fillers are preferred which have a BET surface area of 50 to 300 m²/g, in particular 100 to 200 m²/g, and particularly preferably 120 to 180 m²/g. It was found that, for the given particle size, fillers with a surface area in this range achieve a good compromise between an improvement of the mechanical properties and an increase of the thickening effect.

The BET surface area is determined in accordance with DIN ISO 9277 with a NOVA 2000e BET determination instrument (Quantachrome, now Anton Paar, Graz, Austria). Nitrogen with a temperature of about 77 K is used as adsorbate. The gaseous adsorbate is added to the sample container which is kept at constant temperature. The adsorbate quantities which are taken up are measured in equilibrium with the gas pressure p of the adsorbent and are plotted against the relative pressure p/p0 as adsorption isotherm. This allows the specific surface area and pore volume distribution to be determined. Sample processing is performed by bake-out or drying at 250° C. for 1 or 3 h, respectively. A 9 mm measuring cell is used.

In a preferred embodiment, the dental materials according to the invention contain a mixture of two or more fillers, in particular two or more fillers with different particle sizes. It was found that the use of such filler mixtures does not excessively increase the viscosity of the materials and that the compositions can therefore readily be processed by additive processes, such as stereolithography. The total filler content is preferably in the range of from 0.1 to 20 wt.-%, particularly preferably from 2 to 10 wt.-%.

SiO₂-based fillers can be surface modified with methacrylate-functionalized silanes to improve the bond between the filler particles and the crosslinked polymerization matrix. A preferred example of such silanes is 3-methacryloyloxypropyltrimethoxysilane. It is further preferred that fillers which are surface-modified with methacrylate-functionalized silanes, in particular with 3-methacryloyloxypropyltrimethoxysilane, have a carbon content of 3 to 8 wt.-%, in particular 4.5 to 6.5 wt.-%, determined in accordance with ISO 3262-20.

Functionalized acid phosphates, such as 10-methacryloyloxydecyl dihydrogen phosphate, can also be used for surface modification of non-silicate fillers such as ZrO₂ or TiO₂.

Unless otherwise stated, all particle sizes herein are weight-average particle sizes, wherein the determination of particle sizes in the range of 0.1 μm to 1000 μm is effected by means of static light scattering, preferably using an LA-960 static laser scattering particle size analyzer (Horiba, Japan). Here, a laser diode with a wavelength of 655 nm and an LED with a wavelength of 405 nm are used as light sources. The use of two light sources with different wavelengths allows the entire particle size distribution of a sample to be measured in only one measurement run, wherein the measurement is carried out as a wet measurement. For this purpose, a 0.1 to 0.5% aqueous dispersion of the filler is prepared and the scattered light thereof is measured in a flow cell. The scattered light analysis for calculating particle size and particle size distribution is carried out in accordance with to the Mie theory as per DIN/ISO 13320.

Particle sizes smaller than 0.1 μm are preferably determined by means of dynamic light scattering (DLS). For this purpose, 1 g of the filler, which is to be determined, is taken up in 50 g water and 3 ml of 20% sodium polyphosphate solution is added. The particle size in the range from 5 nm to 0.1 μm is preferably measured by dynamic light scattering (DLS) of aqueous particle dispersions, preferably with a Nanotrac Flex particle sizer (Microtrac Retsch GmbH/Haan, Germany) using a He—Ne laser with a wavelength of 780 nm (intensity 3 mW), at a measuring angle of 180° at 25° C. The results are calculated by evaluating the autocorrelation function.

The materials according to the invention preferably contain a maximum of 5 wt.-%, in particular a maximum of 3 wt.-%, particularly preferably a maximum of 0.3 wt.-% and further preferred no filler particles, such as primary particles, agglomerates and aggregates, with an average particle size of more than 350 nm. Coarser fillers, such as glass powder, are conventionally used to obtain materials with good mechanical properties. They are used in particular for the preparation of replacement teeth, because these are subjected to high stresses during chewing. Coarse fillers, however, are not suitable for the preparation of denture components because their use causes problems in the processing of the materials, e.g. because of their tendency to sediment and an increased susceptibility to discolorations. On the other hand, fine-particle fillers have the disadvantage that, due to their higher thickening effect, they can only be used in relatively small amounts, which is disadvantageous with regard to the mechanical properties. In addition, high viscosity makes processing with 3D printers more difficult.

According to the invention, it was found that dental materials suitable for the preparation of both denture base and replacement teeth can be obtained by combining nanoparticulate or microfine filler, which have an average primary particle size in the range of from 10 nm to 350 nm, and specific ABA triblock copolymers.

Photoinitiators (e) that are preferred according to the invention for initiating radical photopolymerization are benzophenone, benzoin and their derivatives as well as α-diketones and their derivatives, such as 9,10-phenanthrenequinone, 1-phenylpropane-1,2-dione, diacetyl or 4,4′-dichlorobenzil. Particularly preferred are camphorquinone (CQ) and 2,2-dimethoxy-2-phenyl-acetophenone, in particular α-diketones in combination with amines as reducing agents, such as 4-(dimethylamino)-benzoic acid ester (EDMAB), N,N-dimethylaminoethyl methacrylate, N,N-dimethyl-sym.-xylidine or triethanolamine.

Further particularly preferred photoinitiators are Norrish type I photoinitiators, in particular monoacyl- or bisacyl phosphine oxides, such as diphenyl(2,4,6-trimethylbenzoyl)phenylphosphine oxide (CAS No. 75980-60-8), and monoacyltrialkyl-diacyldialkyl- or tetraacylgermanium compounds, such as benzoyltrimethylgermanium, dibenzoyldiethylgermanium, bis(4-methoxybenzoyl)diethylgermanium (MBDEGe), tetrakis(4-ethoxybenzoyl)germanium or tetrakis(4-propoxybenzoyl)germanium, or acyltin compounds, such as monoacylstannanes, diacylstannanes, triacylstannanes or tetraacylstannanes, such as e.g. benzoyltriphenyltin. Further preferred acylgermanium compounds are described in EP 3 150 641 A1 and further preferred acyltin compounds in EP 3 293 215 A1. Mixtures of the various photoinitiators can also be used advantageously, such as bis(4-methoxybenzoyl)diethylgermanium in combination with camphorquinone and 4-dimethylaminobenzoic acid ethyl ester.

Particularly preferred are camphorquinone (CAS No. 10373-78-1) in combination with ethyl 4-(dimethylamino)benzoate (EMBO, CAS No. 10287-53-3) as well as phenylbis(2,4,6-trimethylbenzoyl) phosphine oxide (BAPO, CAS 162881-26-7), diphenyl(2,4,6-trimethylbenzoyl)phenyl phosphine oxide (TPO, CAS No. 75980-60-8), 2,4,6-trimethylbenzoyldiphenyl phosphinate (TPO-L, CAS No. 84434-11-7), 2-benzyl-2-(dimethylamino)-4′-morpholinobutyrophenone (Irgacure 369, CAS No. 119313-12-1), 1-butanone-2-(dimethylamino)-2-(4-methylphenyl)methyl-1-4-(4-morpholinyl)phenyl(Irgacure 379, CAS No. 119344-86-4) and most particularly bis(4-methoxybenzoyl) diethylgermanium (MBDEGe; Ivocerin), tetrakis(4-ethoxybenzoyl) germanium or tetrakis(4-propoxybenzoyl)germanium. According to the invention, photoinitiators are particularly preferred which either have only a very low intrinsic color or lose their color upon irradiation with light, e.g. in the stereolithographic printing process or in the light-curing process. The most preferred photoinitiators are TPO and BAPO.

In addition to the above-mentioned components, the materials according to the invention can additionally contain one or more additives (f), in particular stabilizers, colorants, plasticizers, thixotropic additives, dispersants, defoamers, microbicidal agents and/or blowing agents.

In a further preferred embodiment, the material according to the invention additionally contains a thermal initiator, preferably a compound which can be brought into a reactive state by heat, in particular benzoyl peroxide. In the case that a thermal initiator is contained in the material, thermal post tempering can be carried out after the radical polymerization to achieve better mechanical properties, preferably at a temperature of at least 80° C., in particular at least 100° C. Particularly preferred are compounds which comprise at least one heat-removable protective group.

The dental materials according to the invention are particularly suitable for the preparation of the denture base and the replacement teeth of full or partial dentures. This does not mean that the compositions used for preparing the denture base and for preparing the replacement teeth are absolutely identical. The essence of the invention is rather to provide a material that can be adapted to the fabrication of denture bases or replacement teeth by minor modifications. The adaptation can be accomplished by varying the type and quantity of components used. Preferably, the variation in the type of the components is effected within the defined groups and, in particular, within the preferred groups. For example, one monomer (a) can be replaced entirely or preferably partially by another monomer (a). According to a preferred embodiment of the invention, the adaptation is carried out by varying the amounts of the components used to prepare the materials, such that the materials for the preparation of the denture base and for the preparation of the replacement teeth contain the same components but in different proportions. For example, it is possible to vary the percentage of block copolymers or to adjust the ratio of components (a) and (b). The use of other or additional components is possible to a minor extent, e.g. alternative monomers of the type of component (b) can be added in an amount of up to 10 wt.-%. For example, materials for preparing the denture base are gingiva-colored and materials for preparing replacement teeth are tooth-colored, and therefore different colorants, in particular pigments, are used. In the simplest case, the adjustment is effected exclusively by coloring the materials accordingly.

The materials according to the invention preferably contain at least one (1) and preferably 2 to 5 colorants. Colorants are preferably used in a concentration of 0.0001 to 0.5 wt.-%. Colorants preferred according to the invention are organic dyes and pigments, in particular azo dyes, carbonyl dyes, cyanine dyes, azomethines and methines, phthalocyanines and dioxazines. Dyes which are soluble in the materials of the invention, in particular azo dyes, are particularly preferred. Particularly preferred are organic and in particular inorganic pigments which are readily dispersible in the dental materials according to the invention. Preferred inorganic pigments are metal oxides or hydroxides, such as titanium dioxide or ZnO as white pigments, iron oxide (Fe₂O₃) as red pigment or iron hydroxide (FeOOH) as yellow pigment. Preferred organic pigments are azo pigments, such as monoazo yellow and orange pigments, diazo pigments or β-naphthol pigments, and non-azo or polycyclic pigments, such as phthalocyanine, quinacridone, perylene and flavanthrone pigments. Azo pigments and non-azo pigments are particularly preferred. The most preferred pigments for coloring both the denture base and other gingiva-colored components as well as the replacement teeth are titanium oxide pigments, iron oxides and azo pigments.

The dental materials according to the invention can contain one or more thixotropic additives. These additives bring about thickening of the materials and can thus, for example, prevent fillers from sedimenting. In particular, filler-containing materials therefore preferably contain at least one thixotropic additive. Preferred thixotropic additives are OH group-containing polymers, such as e.g. cellulose derivatives, and inorganic substances, such as layer silicates. In order not to increase the viscosity of the materials too much, the dental materials according to the invention preferably contain only 0 to 3.0 wt.-%, particularly preferably 0 to 2.0 wt.-% and quite preferably no thixotropic additive, based on the total weight of the material. Certain fillers, such as highly dispersed SiO₂, i.e. SiO₂ with a small primary particle size (<20 nm) and a large surface area (>100 m²), have a thixotropic effect and can replace thixotropic additives.

The rheological properties of the dental materials according to the invention are matched to the desired application. Materials for stereolithographic processing are preferably adjusted such that their viscosity is in the range of 50 mPa·s to 50 Pa·s, preferably 100 mPa·s to 10 Pa·s, particularly preferably 100 mPa·s to 5 Pa·s. The viscosity is determined at 25° C. using a cone-plate viscometer (shear rate 100/s). At 25° C. the dental materials according to the invention particularly preferably have a viscosity<10 Pa·s and quite particularly preferably <5 Pa·s. The viscosity is preferably determined with an Anton Paar MCR 302-type viscometer with a CP25-2 cone-plate measuring system and a measuring gap of 53 μm in rotation at a shear rate of 100/s. Due to their low viscosity, the dental materials according to the invention are particularly suitable for being processed using additive manufacturing processes, such as 3D printing or stereolithography. The processing temperature is preferably in a range of 10 to 70° C., particularly preferably 20 to 30° C.

According to the invention, dental materials with the following composition are particularly preferred:
(a) 30 to 70 wt.-%, preferably 30 to 61 wt.-% and particularly preferably 40 to 60 wt.-% of at least one monofunctional radically polymerizable (meth)acrylate,
(b) 20 to 60 wt.-%, preferably 30 to 55 wt.-% and particularly preferably 33 to 55 wt.-% of at least one polyfunctional radically polymerizable (meth)acrylate,
(c) 0.5 to 16 wt.-%, preferably 1 to 13 wt.-% and particularly preferably 2 to 10 wt.-% of at least one ABA triblock copolymer,
(d) 0.2 to 12 wt. %, preferably 1 to 10 wt.-% and particularly preferably 2 to 8 wt.-% of at least one nanoparticulate or microfine filler,
(e) 0.1 to 5.0 wt.-%, preferably 0.2 to 4.0 wt.-% and particularly preferably 0.3 to 3.0 wt.-% of at least one photoinitiator for radical polymerization, and
(f) 0 to 3 wt.-% of additives, such as pigments.

Unless otherwise stated, all percentages herein refer to the total weight of the material. Component (b) can contain 0 to 35 wt.-%, preferably 0 to 25 wt.-% and particularly preferably 0 to 10 wt.-% of one or more further dimethacrylates, based on the total weight of the material, wherein dental materials which contain no further radically polymerizable polymethacrylates are most preferred.

Surprisingly, it was found that the specific combination of the above components provides dental materials which are suitable for the preparation of denture bases and other gingiva-colored parts of full or partial dentures, as well as for the preparation of the replacement teeth of the dentures.

The triblock copolymers used according to the invention, in combination with the nanoparticulate or microfine fillers and a defined monomer mixture, provide materials that can be matched to the different mechanical requirements of denture bases and replacement teeth, such as toughness, flexural strength and flexural modulus, and different esthetic requirements, such as opacity and coloring, by varying the proportions of the components and by appropriate coloring. Materials with a monomer mixture which contains at least one tricyclic acrylate and at least one polyfunctional urethane dimethacrylate proved to be particularly advantageous, particularly preferably a mixture of at least one monomer of formula II or III and a urethane dimethacrylate, and most preferably a mixture of TCDA and UDMA.

The matched materials are characterized by a similar polymerization shrinkage, a similar water absorption and a good biocompatibility. This is how a strong bond is achieved at the interfaces between denture base and replacement teeth in the case that denture base and replacement teeth are prepared one after the other and then joined together, and also in the case that the materials are processed in parallel, i.e. denture base and replacement teeth are, for example, manufactured together in an inkjet printing process. This is a great advantage with regard to additive manufacturing processes and considerably facilitates the production of dental full or partial dentures with such processes.

The monofunctional methacrylates and, in particular, acrylates of formula II and quite particularly of formula III are characterized by a high flexibility, reactivity and a good miscibility with the other components, i.e. in particular with the monomers of component (b) and with the block copolymers. For example, it was found that TCDMA is readily miscible with monomers of component (b), in particular UDMA, and with the block copolymers.

The dental materials according to the invention are characterized in that they have a good fracture toughness and fracture work and, at the same time, a good flexural strength and a relatively high modulus of elasticity, measured at 37° C. in water, which corresponds to oral conditions. The materials also have a high transparency and low viscosity before curing. Furthermore, it is advantageous for the selected use that the dental materials have a higher opacity after curing than before curing but a low inherent color.

After curing, materials for the preparation of denture bases preferably have a fracture toughness Kₘₐₓ of greater than 1.0 MPa·m^{1/2}, in particular greater than 1.2 MPa·m^{1/2}, particularly preferably greater than 1.4 MPa·m^{1/2} as well as preferably a fracture work FW that is greater than 150 J/m², in particular greater than 175 J/m², particularly preferably greater than 200 J/m². The determination of the fracture toughness Kₘₐₓ and the fracture work FW is effected in accordance with ISO 20795-1:2013 in the 3-point bending test with a support span of 32 mm. Furthermore, the cured materials preferably have a flexural modulus, determined in accordance with ISO20795-1:2013, of at least 1500 MPa, in particular of at least 2000 MPa, particularly preferably of at least 2150 MPa. It is further preferred that after curing the materials have a flexural strength, determined in accordance with ISO20795-1:2013, of at least 50 MPa, in particular at least 60 MPa, and particularly preferably at least 65 MPa.

After curing, the materials for the preparation of replacement teeth preferably have a fracture toughness Kₘₐₓ of greater than 0.8 MPa·m^{1/2}, in particular greater than 1.0 MPa·m^{1/2}, particularly preferably greater than 1.1 MPa·m^{1/2} as well as preferably a fracture work FW that is greater than 100 J/m², in particular greater than 125 J/m², particularly preferably greater than 130 J/m². Furthermore, the cured materials preferably have a flexural modulus, determined in accordance with ISO20795-1:2013, of at least 2200 MPa, preferably of at least 2400 MPa or more, more preferably of at least 2500 MPa. It is further preferred that after curing the materials have a flexural strength, determined in accordance with IS020795-1:2013, of at least 60 MPa, in particular at least 65 MPa, and particularly preferably at least 70 MPa.

In deviation from IS020795-1:2013, a printed specimen is used to determine the fracture toughness Kₘₐₓ, the fracture work FW, the flexural strength and the flexural modulus. The test specimens are prepared with a PrograPrint PR5 stereolithography system (Ivoclar/Schaan, Liechtenstein) using PrograPrint CAM software (Ivoclar/Schaan, Liechtenstein). The test specimens are prepared with a layer height of 100 μm at room temperature (23° C.) with an oversize of 0.1 mm (x-, y-, z-direction) and cleaned after the printing process in a PrograPrint Clean cleaning unit (Ivoclar/Schaan Liechtenstein) for 2 min each at a speed of 850 rpm in the two cleaning baths on the build platform. After cleaning, the test specimens are blown off with oil-free compressed air and light-curing is carried out in the PrograPrint Cure light-curing unit (Ivoclar/Schaan, Liechtenstein) for 90 seconds at a wavelength of 405 nm at 100% power. Then the specimens are ground with P1000 grit wet-grinding paper to the dimension required for the standard tests of IS020795-1 and stored and tested in accordance with the specifications of the standard test.

Parts made from these materials are highly resistant to deformation without breaking.

After curing, the materials according to the invention have a good flexural strength, a good flexural modulus and a good fracture toughness, as well as a good fracture work. Shaped bodies which are obtained by curing the materials according to the invention have high stiffness and offer high levels of resistance to deformation without breaking.

It was found that the preferred compositions of the material according to the invention described above bring about particularly advantageous mechanical properties, wherein the molar ratio of component (a) to component (b) is of particular importance. For denture base materials, the molar ratio of component (a) to component (b) is preferably 1.5 to 2.0:1, in particular 1.6 to 1.9:1. For replacement teeth materials, the ratio is preferably 1.0 to 1.5:1, in particular 1.1 to 1.3:1. In the case that, in addition to this preferred ratio, ABA triblock copolymers in the preferred range are used, the material has a particularly advantageous fracture toughness. Furthermore, the material has particularly advantageous modulus values in the case at least one nanoparticulate or microfine filler is additionally used in the preferred amount.

The invention is also directed to the use of the dental materials described above for the preparation of a dental prosthesis or a part thereof.

The materials according to the invention are suitable to be processed by 3D printing, in particular by stereolithography and 3D inkjet processes. Preferred 3D inkjet processes are multi-material inkjet printing processes.

In stereolithography, a denture base and a dental arch are initially printed. Denture base material or replacement teeth material is applied in a non-polymerized state as an adhesive layer in the cavities provided for the teeth in the denture base. The dental arch is then positioned on the denture base before light is used to polymerize the denture base material or the replacement teeth material of the adhesive layer, creating a stable chemical bonding between the adhesive layer and the denture base or dental arch, respectively. The use of denture base material as the adhesive layer is particularly preferred.

Preferably, dental prostheses are prepared using a stereolithographic process which comprises the steps:
(1) Preparing a denture base in a 3D printing process using a dental material according to the invention described above,
(2) optionally, curing at least partially the denture base obtained in step (1) by radical polymerization,
(3) preparing one or more replacement teeth, in particular an arch of interconnected teeth, in a 3D printing process using a dental material according to the invention described above,
(4) optionally, curing at least partially the replacement tooth or teeth obtained in step (3) by radical polymerization,
(5) optionally, applying an adhesive to a surface of the denture base obtained in step (2) and/or to a surface of the replacement tooth or teeth obtained in step (4),
(6) joining the denture base with the replacement tooth or teeth,
(7) curing the product obtained in step (6) by radical polymerization,
(8) optionally a thermal treatment.

The optional thermal treatment can be advantageous for improving the mechanical properties and, in particular, can result in complete curing.

In one embodiment, the denture base has one or more cavities into which the replacement teeth can be inserted. The surface of the cavity or of the cavities is preferably only partially cured and is cured completely only after the insertion of the replacement tooth or teeth.

In multi-material inkjet printing processes, the denture base material and the replacement teeth material can be joined together during the printing process, which means that, in contrast to stereolithography, after printing no additional process step is required for joining the components together. A first layer of denture base material is initially printed until, at a digitally defined position, e.g. within the layer or at the transition to the next layer, the material is changed and the printing process is continued with the replacement teeth material. The different materials are usually printed with different print heads. In this way, a transition between the different materials is formed during the printing process. The order in which denture base material and the replacement teeth material are printed can be switched back and forth as desired. Due to the very similar material properties, the two materials, which are chemically very similar but optically and possibly also with regard to the mechanical properties different, crosslink during the printing process and form a particularly strong bond.

Preferably, dental prostheses are prepared using a multi-material inkjet printing process which comprises the steps:
(1) depositing a layer consisting of a denture base material, a dental material or a combination thereof in a layerwise manner using an inkjet printer,
(2) curing the applied material during or after, preferably after, the application of the layer formed in step (1) at least partially, wherein the curing is preferably effected by UV light,
(3) repeating steps (1) and (2) in order to obtain a body with the desired geometric shape and coloring.

The invention also relates to denture bases, replacement teeth, full or partial dentures which are prepared using the dental materials of the invention.

The invention is explained in more detail in the following with reference to examples.

### EXAMPLES

### Example 1

Production of Dental Materials

The components listed in Tables 1 were homogeneously mixed with each other in the indicated amounts. For this purpose, all solid components (block copolymer or core-shell particles, photoinitiator) were dispersed or dissolved in the monomers using a magnetic stirrer, if necessary also by heating to 60° C. Then, the urethane dimethacrylate was added and stirring continued until a homogeneous mixture was obtained. The block copolymers were easily incorporated into the mixtures without the need for a rotor-stator dispersing system or a similar system. The mixture was then degassed by means of a speed mixer (Hauschild GmbH in Hamm/Germany).

TABLE 1
Compositions of materials for the preparation
of denture bases and replacement teeth

| | Ex. 1* | Ex. 2* | Ex. 3 | Ex. 4* | Ex. 5* | Ex. 6 |
|---|---|---|---|---|---|---|
| Component | % | % | % | % | % | % |
| (a) TCDMA²⁾ | 32.00 | 41.00 | 39.50 | 34.50 | 32.75 | 31.00 |
| (b) UDMA | 59.50 | 50.50 | 48.50 | 64.50 | 61.25 | 58.00 |
| Molar ratio a:b | 1.15:1 | 1.73:1 | 1.74:1 | 1.14:1 | 1.14:1 | 1.14:1 |
| (d) Aerosil R 711³⁾ | — | — | 4.00 | — | — | 5.00 |
| (c) Block copolymer⁴⁾ | 7.50 | 7.50 | 7.00 | — | 5.00 | 5.00 |
| (e) TPO⁵⁾ | 1.00 | 1.00 | 1.00 | 1.00 | 1.00 | 1.00 |
| Fracture toughness [MPa] | 72.4 | 62.0 | 67.8 | 91.6 | 83.8 | 71.4 |
| E-modulus [MPa] | 1776 | 2010 | 2174 | 2561 | 2432 | 2239 |
| Kₘₐₓ [MPa · m^{1/2}] | 1.27 | 1.37 | 1.45 | 0.62 | 1.05 | 1.11 |
| Fracture Work [J/m²] | 223 | 311 | 282 | 45 | 124 | 131 |

| | | |
|---|---|---|
| Indication | Denture base material | Replacement teeth material |

*Comparative example
²⁾A tricyclodecane methanol monoacrylate of formula III
³⁾Fumed silica which is, according to the manufacturer (Evonik Operations GmbH), functionalized with methacrylate-functionalized silanes and has a carbon content of 4.5 to 6.5 wt.-%.
⁴⁾PCL-b-PDMS-b-PCL block copolymer, the molar mass of PDMS blocks is 3200 g/mol and the molar mass of PCL blocks is 1600 g/mol.
⁵⁾Diphenyl(2,4,6-trimethylbenzoyl)phenyl phosphine oxide (CAS No. 75980-60-8).

All examples exhibited a modulus of elasticity of at least 2000 MPa and a flexural strength of at least 65 MPa. Thus, all materials according to the invention exhibited the required mechanical properties for the preparation of dental prostheses. In addition, the materials according to the invention exhibited a fracture toughness of more than 1 MPa·m^{1/2} and a fracture work of more than 100 J/m², which is also advantageous for use as a denture material. Numerous known prosthesis materials for stereolithography, which consist for example only of dimethacrylates, exhibit inferior mechanical properties, such as a fracture toughness below 1 MPa·m^{1/2} or a fracture work of less than 100 J/m².

It was further found that the denture base material of Example 3 and the replacement teeth material of Example 6 are particularly suitable for use in additive processes, such as stereolithography and inkjet printing processes, and for being bonded together. It was found that a particularly strong bond between the materials is formed in these processes.

1. A radically polymerizable material for the preparation of dental prostheses, comprising
(a) at least one monofunctional radically polymerizable (meth)acrylate,
(b) at least one polyfunctional radically polymerizable (meth)acrylate,
(c) at least one block copolymer,
(d) at least one nanoparticulate or microfine filler having an average primary particle size from 10 nm to 350 nm, and
(e) at least one photoinitiator,
characterized in that
the block copolymer (c) is an ABA triblock copolymer.

2. The dental material according to claim 1, which comprises as component (c) a silicon-containing ABA triblock copolymer, wherein the A block is an oligomeric polyester or a poly(meth)acrylate, and the B block is an oligomeric polysiloxane.

3. The dental material according to claim 2, which comprises as component (c) an ABA triblock copolymer of the type PCL-b-PDMS-b-PCL and/or polymethyl methacrylate PMMA-b-PDMS-b-PMMA, wherein the molar ratio of A:B is in the range of from 0.1 to 5 and the molar mass of the ABA triblock copolymer is in the range of from 3 to 25 kDa.

4. The dental material according to claim 1, wherein component (c) comprises 60:40 to 40:60 wt.-% A blocks and 40:60 to 60:40 wt.-% of the B block.

5. The dental material according to claim 1, wherein component (a) comprises a non-aromatic alicyclic, bicyclic or tricyclic methacrylate or acrylate, of the following formula II: 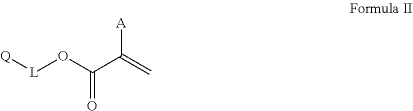
in which
A is H or methyl,
L is a chemical bond, C₁-C₃ alkylene or —(CR¹R²—CR³R⁴—O)ₘ—, wherein R¹ to R⁴ are each independently H or a C₁-C₃alkyl group, and m is an integer from 2 to 10, and
Q is one of the following groups in which • represents the site of linkage to L:

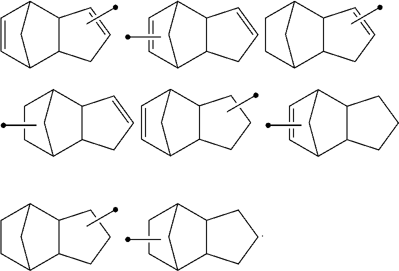

6. The dental material according to claim 5, wherein component (a) comprises a tricyclodecane methanol monoacrylate having the following formula III:

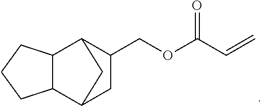 [SMILES: C=CC(=O)OCC1CC2CC1C1CCCC21]

7. The dental material according to claim 1, wherein component (b) comprises a polyfunctional urethane (meth)acrylate, a urethane methacrylate or a urethane dimethacrylate (UDMA).

8. The dental material according to claim 1, wherein component (d) comprises SiO₂, ZrO₂, TiO₂, a mixed oxide of SiO₂, ZrO₂, ZnO and/or TiO₂, fumed or precipitated silica.

9. The dental material according to claim 8, wherein component (d) has a BET surface area of 50 to 300 m²/g.

10. The dental material according to claim 1, which comprises
(a) 30 to 70 wt.-% of at least one monofunctional radically polymerizable (meth)acrylate,
(b) 20 to 60 wt.-% of at least one polyfunctional radically polymerizable (meth)acrylate,
(c) 0.5 to 16 wt.-% of at least one ABA triblock copolymer,
(d) 0.2 to 12 wt.-% of at least one nanoparticulate or microfine filler,
(e) 0.1 to 5.0 wt.-% of at least one photoinitiator for radical polymerization, and
(f) 0 to 3 wt.-% of additives,
in each case relative to the total mass of the material.

11. The dental material according to claim 10, which comprises as component (b) to 60 wt.-% or 30 to 55 wt.-% or 33 to 55 wt.-% of at least one urethane dimethacrylate and 0 to 35 wt.-% or 0 to 25 wt.-% or 0 to 10 wt.-% of further di(meth)acrylates, in each case relative to the total mass of the material.

12. A process of using a dental material according to claim 1 for preparation of a dental prosthesis or a part thereof.

13. The process according to claim 12, wherein the preparation of the dental prosthesis or part thereof is performed by 3D printing or stereolithography.

14. The process according to claim 12, wherein the preparation of the dental prosthesis is performed by a 3D inkjet process or a multi-material inkjet printing process.

15. A process for preparing a dental prosthesis comprising the steps of:
(1) preparing a denture base in a 3D printing process using a dental material according to claim 1,
(2) optionally, curing at least partially the denture base obtained in step (1) by radical polymerization,
(3) preparing one or more replacement teeth in a 3D printing process using a dental material according to claim 1,
(4) optionally, curing at least partially the replacement tooth or teeth obtained in step (3) by radical polymerization,
(5) optionally, applying an adhesive to a surface of the denture base obtained in step (2) and/or to a surface of the replacement tooth or teeth obtained in step (4),
(6) joining the denture base with the replacement tooth or teeth,
(7) curing the product obtained in step (6) by radical polymerization,
(8) optionally applying a thermal treatment.
